(19) United States
(12) Patent Application Publication
(10) Pub. No.: US 2011/0152994 A1
(43) Pub. Date: Jun. 23, 2011

(54) IMPLANTABLE DEVICE

(75) Inventors: Per Hendriksen, Herlufmagle (DK); Palle M. Hansen, Bjaeverskov (DK)

(73) Assignees: William Cook Europe ApS, Bjaeverskov (DK); Cook Incorporated, Bloomington, IN (US)

(21) Appl. No.: 12/963,301

(22) Filed: Dec. 8, 2010

(30) Foreign Application Priority Data
Dec. 22, 2009 (GB) 0922456.9

(51) Int. Cl. A61F 2/84; A61F 2/82; C22C 14/00

(52) U.S. Cl. 623/1.11; 623/1.15; 623/1.16; 420/417

(57) ABSTRACT

An intracranial stent formed from Nitinol has variable flexibility. The stent (10) is formed from a plurality of stent rings (12) connected by tie bars (14). The tie bars (14) in a central soft zone (16) are heat treated so as to confer a higher transition temperature on the Nitinol. As a result, at body temperature, the soft zone (16) has reduced longitudinal stiffness as compared to the stiffer zones (18) found at the ends of the stent (10).

### TECHNICAL FIELD

The present application relates to an implantable device, such as a stent, in particular for use as an intracranial implant or in other delicate vessels and organs. It also relates to an introducer or catheter assembly including an implantable device as taught herein.

### BACKGROUND OF THE INVENTION

Stents are used for treatment of vasculature in the human or animal body normally to prevent or counteract an illness or a disease-induced localised flow constriction. Surgical stents have long been known which can be surgically implanted into a body lumen, such as in an artery, to reinforce, support, repair or otherwise enhance the performance of the lumen. For instance, in cardiovascular surgery it is often desirable to place a stent in the coronary artery at a location where the artery is damaged or is susceptible to collapse. The stent, once in place, reinforces that portion of the artery allowing normal blood flow to occur through the artery. One form of stent which is particularly desirable for implantation in arteries and other body lumens is a tubular stent which is formed as a complete tubular cylinder and can be radially expanded from a first smaller diameter to a second larger diameter. Such radially expandable stents can be inserted into the artery by being located on a catheter and fed internally through the arterial pathways of the patient until the unexpanded stent is located where desired. The catheter may be fitted with a balloon or other expansion mechanism which exerts a radial pressure outwardly on the stent causing the stent to expand radially to a larger diameter. Such expandable stents exhibit sufficient rigidity after being expanded that they will remain expanded after the catheter has been removed.

Stents may also be self-expanding. For example, a stent may be formed from a shape-memory alloy such as Nitinol (nickel titanium alloy). Nitinol is able to transform from a Martensitic phase to an Austenitic phase by being heated. The temperature at which this transformation occurs (the “transition temperature” or “Austenite finish temperature”) depends on the composition of the alloy. The transition temperature can be altered by heat treatment.

Below its transition temperature, Nitinol is in its Martensitic phase and can be deformed into a new shape. For example, a stent may be compressed ready for deployment. At its transition temperature, Nitinol transforms into its Austenitic phase and reverts to its original shape. In the case of a stent, therefore, the stent will expand from its compressed state into its expanded state. The transition temperature of Nitinol usually used in stents is therefore below body temperature, so that at body temperature the stent is at a temperature above its transition temperature and the stent expands.

Above its transition temperature, Nitinol exhibits super-elasticity. Therefore, to remain compressed at body temperature a Nitinol stent must be held in its compressed configuration by means of, for example, restraining wires or some other constraining mechanism.

Narrowing of the arteries inside the skull is a significant cause of stroke. Whilst angioplasty including stent placement is commonly carried out in major blood vessels, special considerations need to be taken into account for intracranial stent placement. The arteries of the brain are particularly delicate, and tend to be considerably more tortuous than the vasculature elsewhere in the body.

Stents are disclosed in U.S. Pat. No. 6,652,576, U.S. Pat. No. 6,485,507, U.S. Pat. No. 6,287,336, U.S. Pat. No. 6,764,506, U.S. Pat. No. 6,746,475, U.S. Pat. No. 6,019,789, U.S. Pat. No. 6,652,576, U.S. Pat. No. 6,287,336 and U.S. Pat. No. 6,485,507.

### SUMMARY OF THE INVENTION

According to a first aspect of the present invention, there is provided an implant, wherein the implant includes at least one relatively soft zone flanked by two relatively stiffer zones, wherein the relatively soft zone comprises a region of the implant having reduced longitudinal stiffness compared to the relatively stiffer zones, and wherein the relatively soft zone and the relatively stiffer zones have substantially the same radial stiffness.

Providing a relatively soft zone having a reduced longitudinal stiffness allows the implant to conform more easily to curvature within a tortuous vessel. By flanking the relatively soft zone by relatively stiffer zones, improved security of positioning of the implant within the vessel can be achieved.

In a preferred embodiment, the implant includes a stent. However, the implant could be any other implantable device.

In an embodiment the stent includes a plurality of stent rings connected to each other by tie bars. The relatively soft zone may include relatively soft tie bars and relatively stiff stent rings. This is a preferred way of allowing reduced longitudinal stiffness whilst maintaining radial stiffness.

Preferably a relatively soft zone is located in a substantially central region of the implant. By maintaining longitudinal stiffness towards the ends of the implant, more secure anchoring of the implant within a vessel can be obtained.

Preferably, the implant includes a structure formed from a shape memory material, wherein the relatively stiffer zones include a shape memory material having a transition temperature below normal body temperature, and wherein the relatively soft zone includes a shape memory material having a transition temperature above normal body temperature.

In the preferred embodiment, the implant is a stent including a plurality of stent rings connected to each other longitudinally by tie bars, wherein the tie bars of the relatively soft zone have a transition temperature above normal body temperature, and wherein the stent ring or stent rings of the relatively soft zone have a transition temperature below normal body temperature. This allows reduced longitudinal stiffness within the relatively soft zone whilst maintaining radial stiffness.

The tie bars in the relatively soft zone may have a transition temperature of at least 40° C. In the preferred embodiment the transition temperature is approximately 50° C. Preferably, the tie bars in the relatively stiffer zone have a transition temperature of approximately 25° C.

A relatively stiffer zone may be located at each end of the implant. This assists with secure positioning of the implant within a vessel. Each relatively stiffer zone preferably includes at least two stent rings. In an embodiment, each relatively stiffer zone may include up to five stent rings, for example.

Each relatively stiffer zone may comprise 10 to 20% of the length of the implant. In an embodiment, each stiffer zone may comprise 10 to 15% of the length of the implant.

According to a second aspect of the present invention there is provided an introducer having an implant as described above loaded thereon.

According to a third aspect of the present invention there is provided a method of making an implant including at least one relatively soft zone flanked by two relatively stiffer zones, wherein the relatively soft zone comprises a region of the implant having reduced longitudinal stiffness as compared to the relatively stiffer zones, and wherein the relatively soft zone and the relatively stiffer zones have substantially the same radial stiffness, the method including: (a) providing an implant including a structure formed from a shape memory material having a transition temperature below normal body temperature; and (b) heat treating a portion of the implant to form the relatively soft zone.

Preferably the implant is a stent including a plurality of stent rings connected to each other by tie bars, and the tie bars in the relatively soft zone are heat treated to raise their transition temperature to above normal body temperature.

### BRIEF DESCRIPTION OF THE DRAWING

Preferred embodiments of the present invention are described below by way of example only, with reference to the accompanying drawings, in which:

FIG. 1 shows a side view of an embodiment of a stent;

FIG. 2 illustrates the return force at different temperatures of different parts of the stent of FIG. 1;

FIG. 3 illustrates the stent of FIG. 1 located within a curved vessel; and

FIG. 4 illustrates the stent of FIG. 1 loaded onto a deployment device.

### DETAILED DESCRIPTION

It is to be understood that the Figures are schematic and do not show the various components to their actual scale. In many instances, the Figures show scaled up components to assist the reader.

In this description, when referring to an introducer or a deployment assembly, the term distal is used to refer to an end of a component which in use is furthest from the surgeon during the medical procedure, including within a patient. The term proximal is used to refer to an end of a component closest to the surgeon and in practice in or adjacent an external manipulation part of the deployment or treatment apparatus.

On the other hand, when referring to an implant such as a stent or stent graft, the term proximal refers to a location that in use is closest to the patient's heart, in the case of a vascular implant, and the term distal refers to a location furthest from the patient's heart.

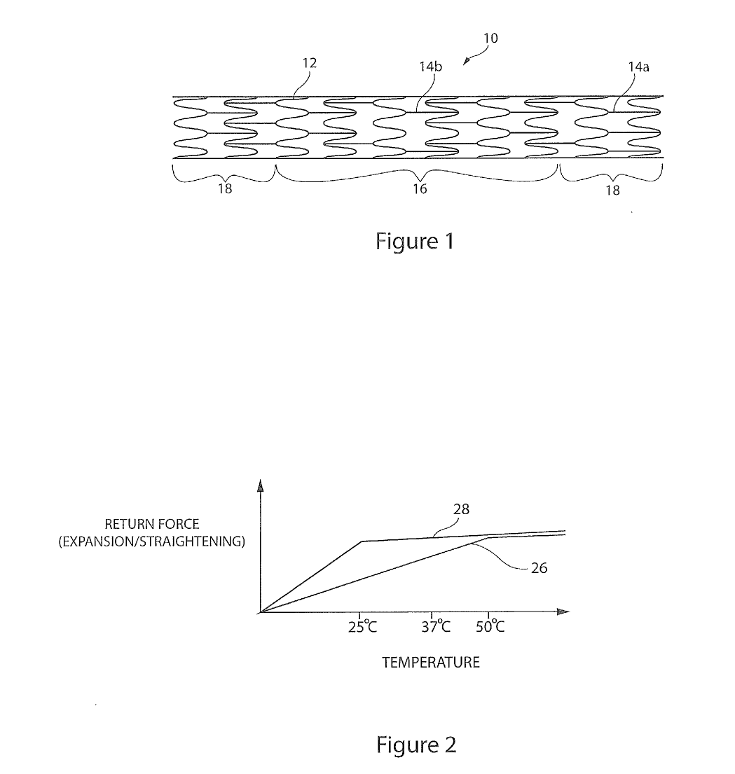

FIG. 1 illustrates an intracranial stent in accordance with a preferred embodiment of the present invention. The stent 10 is formed from a series of Nitinol stent rings 12, in this example there being ten stent rings 12 forming the stent 10. Each stent ring 12 is joined to an adjacent stent ring 12 by means of tie bars 14.

The stent 10 comprises a relatively “soft” zone 16 made up of, in this example, six stent rings 12. This is located between two relatively “stiffer” zones 18 each made up of two stent rings 12 in this embodiment. Each “stiffer” zone 18 is located at an end of the stent 10.

In this embodiment, the stent rings 12 are all formed from Nitinol having a transition temperature of approximately 25° C. As such, the Nitinol of the stent rings 12 is in its Martensitic phase at room temperature, allowing the stent 10 easily to be compressed for deployment and for the stent to retain its compressed configuration. At normal body temperature (approximately 36.8° C.) the Nitinol of the stent rings 12 is above its transition temperature, and is thus in its Austenitic phase. This allows the stent rings 12 to expand when the stent 10 is exposed to normal body temperature and to become biased to return to their shape memory configuration. Once any restraining mechanism, such as restraining wires and/or a sheath, has been removed the stent rings 12 can revert to their expanded state. As a result of this phase transition these parts of the stent exhibit an increased stiffness compared to the characteristics of the stent when it is in its Martenistic phase, in which it is very pliable and thus “soft”.

The tie bars 14b located in the “soft” zone 16 are made from Nitinol having a transition temperature of approximately 50° C. (in practice treated to have a higher transition temperature as described below). This is higher than normal body temperature, and so even once the stent 10 has been deployed, the Nitinol of the tie bars 14b in the “soft” zone 16 remains in its Martensitic phase and can thus be stably deformed. By contrast, the tie bars 14a found in the “stiffer” zones 18 are made from Nitinol having a transition temperature equal to that of the stent rings 12.

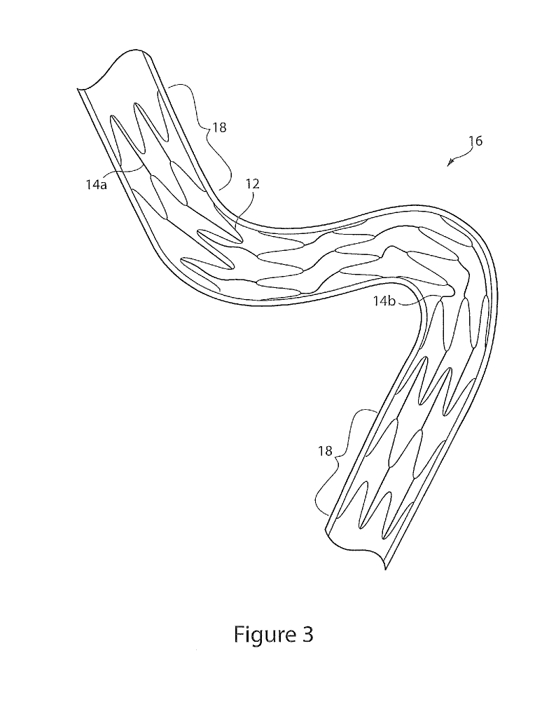

FIG. 2 illustrates the effect of this. Plot 26 illustrates the return force experienced by Nitinol having a transition temperature of approximately 50° C. (as used in the tie bars 14b found in the “soft” zone 16). Plot 28 illustrates the return force experienced by Nitinol having a transition temperature of 25° C. (as utilised in the tie bars 14a in the “stiffer” zones 18). It can be seen that at normal body temperature, there is a difference in the return force of the Nitinol tie bars 14 in the different zones 16, 18 of the stent 10. As a result, the soft zone 16 of the stent 10 is able more easily to conform to the curves of the tortuous brain vasculature. Advantageously, the stent rings 12, even in the soft zone 16, are made from Nitinol having a transition temperature below body temperature in order that the stent 10 retains its radial stiffness in the soft zone 16 so as to provide effective opening of stenosed blood vessels.

From the above it can therefore be seen that the “stiffer” zone 18 exhibits both longitudinal and radial stiffness, whereas the “soft” zone 16 retains radial stiffness substantially equal to that of the “stiffer” zone, but a reduced longitudinal stiffness. The effect of this is that the stent 10 provides a suitable radial force on the walls of a vessel along its entire length. However, the “soft” zone 16 is more easily able to conform to tortuous curvature of a vessel (see FIG. 3).

It is preferred that a “stiffer” region 18 is provided at each end of the stent. This assists with secure location of the stent 10 within the vasculature thereby helping to prevent unwanted migration. As illustrated in FIG. 3, in this example the “stiffer” zone 18 is located in a relatively straight region of the vasculature, because reduced longitudinal stiffness confers little advantage to a region of a stent 10 located within relatively straight vasculature. This assists in providing secure positioning of the stent 10. The skilled person will appreciate that in a preferred embodiment, the “stiffer” zones 18 are relatively short and are dimensioned to fit properly within a vessel to provide secure anchoring. Preferably the “stiffer” zones 18 are not so long as to affect the curvature of the vessel within which the stent has been placed.

The stent 10 is assembled in a conventional manner. In order to create the “soft” zone 16, the tie bars 14b of the “soft” zone 16 are heat treated. Localised heat treating of the tie bars 14b of the “soft” zone 16 may be achieved by any suitable method. The skilled person will appreciate that there are many suitable methods. For example, electrical resistance heating or laser treatment may be used. Further methods include applying a heated inert gas jet or use of an induction coil. Treatment may be carried out at a temperature of about 550° C. to about 600° C. for about 5 minutes to 20 minutes, for example. The precise conditions will depend on the material in question and can be readily determined by the skilled person.

As can be seen in FIG. 3, the tie bars 14b of the soft zone 16, are able to deform plastically (following curvature of the vessel) to allow the stent 10 to curve along its longitudinal axis. However, as the stent rings 12 are above their transition temperature, they will regain their Austenite phase and press outwardly again against the vessel walls. In this way, the stent 10 rings 12 keep the vessel's patency yet ensure that no strain is imparted to the vessel wall in a longitudinal direction.

There may be any suitable number of soft zones 16 and stiffer zones 18. Furthermore, their relative lengths may be altered to suit different purposes. The stiffer end sections 18 are of a dimension to keep the ends of the stent 10 properly aligned in the vessel and maintained in position. For this purpose, the stiffer end sections 18 are preferably of sufficient length to maintain their orientation in the vessel. For this purpose, each stiffer end section 18 is preferably formed of at least two stent rings 12 connected by tie bars 14a in the same Austenite phase at body temperature as the stent rings. It has been found that any number from two to five stent rings 12 (for example, two, three, four or five stent rings 12) per end section 18 provides the optimum end stability and retention with optimal stent flexibility.

The stent 10 might be 40 mm in length and have a 30 mm central soft zone 16 flanked by two 5 mm stiffer zones 18. Preferably, however, each stiffer zone 18 comprises 10 to 20% or 10 to 15% of the length of the stent 10.

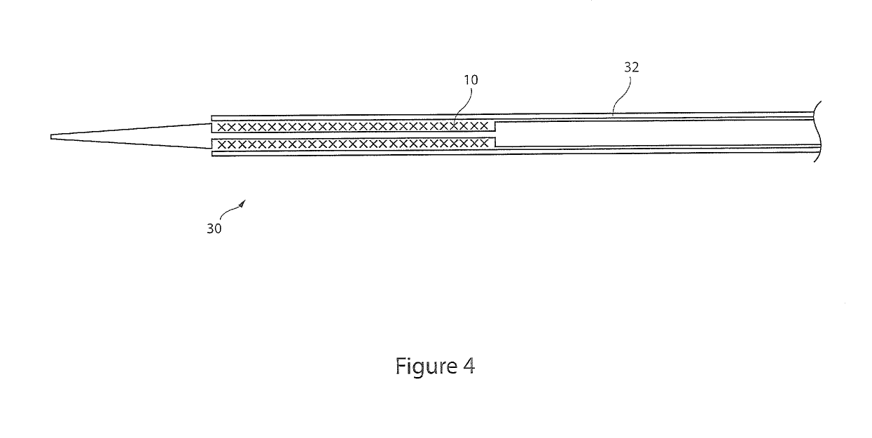

In use, the stent 10 is compressed whilst the Nitinol of all parts of the stent 10 is in its Martensitic phase (at room temperature). FIG. 4 illustrates the compressed stent 10 mounted on a deployment device 30 and covered by a sheath 32. The deployment device is used to deliver the stent 10 to its desired deployment location. The stent 10 is exposed to normal body temperature, which, once the sheath 32 has been withdrawn, causes the stent rings 12 and the tie bars 14 of the hard zones 18 to transform to their original (non-compressed) shape. The Nitinol of the tie bars 14b of the soft zone 16 remains in its Martensitic phase. The tie bars 14b of the soft zone 16 thus remain very flexible at body temperature.

In the above-described embodiment, the Nitinol of the stent rings 12 of the stiffer zone 18 and the soft zone 16 has the same transition temperature and the stent rings 12 thus exhibit the same radial expansion force. However, in alternative embodiments it may be desirable to vary the radial stiffness of the stent rings of different zones.

Many other modifications may be made to the above-described embodiment. In certain embodiments, it might be desirable to flank the stiffer zones 18 with a further soft zone 16 such that the very ends of the stent 16 are soft. This could help to prevent restenosis.

Although preferred embodiments of this invention have been particularly described with reference to a stent, the skilled person will appreciate that the principles described herein could apply equally to other implantable medical devices such as stent grafts, occlusion devices, or filters, for example.

What has been described and illustrated herein is a preferred embodiment of the invention along with some of its variations. The terms, descriptions and Figures used herein are set forth by way of illustration only and are not meant as limitations. Those skilled in the art will recognize that many variations are possible within the spirit and scope of the invention, which is intended to be defined by the following claims, and their equivalents, in which all terms are meant in their broadest reasonable sense unless otherwise indicated.

The features of the various embodiments described above and their modifications may be substituted for or combined with one another as desired. It is also to be understood that the various features of the dependent claims appended hereto may be used with one another in any desired combination of those claims.

What is claimed is:

1. An implant, wherein the implant includes at least one relatively soft zone flanked by two relatively stiffer zones, wherein the relatively soft zone comprises a region of the implant having reduced longitudinal stiffness as compared to the relatively stiffer zones, and wherein the relatively soft zone and the relatively stiffer zones have substantially the same radial stiffness.

2. An implant as claimed in claim 1, wherein the implant includes a stent.

3. An implant as claimed in claim 2, wherein the stent includes a plurality of stent rings connected to each other by tie bars, wherein the relatively soft zone includes relatively soft tie bars and relatively stiff stent rings.

4. An implant as claimed in claim 1, wherein a relatively soft zone is located in a substantially central region of the implant.

5. An implant as claimed in claim 1, wherein the implant includes a structure formed from a shape memory material, wherein the relatively stiffer zones include a shape memory material having a transition temperature below normal body temperature, and wherein the relatively soft zone includes a shape memory material having a transition temperature above normal body temperature.

6. An implant as claimed in claim 5, wherein the implant is a stent including a plurality of stent rings connected to each other longitudinally by tie bars, wherein the tie bars of the relatively soft zone have a transition temperature above normal body temperature, and wherein the stent ring or stent rings of the relatively soft zone have a transition temperature below normal body temperature.

7. An implant as claimed in claim 6, wherein the tie bars in the relatively soft zone have a transition temperature of at least 40° C.

8. An implant as claimed in claim 7, wherein the tie bars in the relatively soft zone have a transition temperature of approximately 50° C.

9. An implant as claimed in claim 7, wherein the tie bars in the relatively stiffer zone have a transition temperature of approximately 25° C.

10. An implant as claimed in claim 1, wherein a relatively stiffer zone is located at each end of the implant.

11. An implant as claimed in claim 10, wherein each relatively stiffer zone located at the ends of the implant includes at least two stent rings.

12. An implant as claimed in claim 10, wherein each relatively stiffer zone located at the ends of the implant includes up to five stent rings.

13. An implant as claimed in claim 10, wherein the relatively stiffer zones each comprise 10 to 20% of the length of the implant.

14. An implant as claimed in claim 12, wherein the relatively stiffer zones each comprise 10 to 15% of the length of the implant.

15. An implant as claimed in claim 1, wherein the implant is an intracranial implant.

16. An implant as claimed in claim 1, wherein the implant is an intracranial stent.

17. An introducer having an implant as claimed in claim 1 loaded thereon.

18. A method of making an implant including at least one relatively soft zone flanked by two relatively stiffer zones, wherein the relatively soft zone comprises a region of the implant having reduced longitudinal stiffness as compared to the relatively stiffer zones, and wherein the relatively soft zone and the relatively stiffer zones have substantially the same radial stiffness, the method including:
(a) providing an implant including a structure formed from a shape memory material having a transition temperature below normal body temperature; and
(b) heat treating a portion of the implant to form the relatively soft zone.

19. A method as claimed in claim 18, wherein the implant is a stent including a plurality of stent rings connected to each other by tie bars, and wherein the tie bars in the relatively soft zone are heat treated to raise their transition temperature to above normal body temperature.
